(19) United States
(12) Patent Application Publication
(10) Pub. No.: US 2013/0190642 A1
(43) Pub. Date: Jul. 25, 2013

(54) APPARATUS AND METHOD FOR DIAGNOSING OBSTRUCTIVE SLEEP APNEA

(71) Applicants: Guido Muesch, Erkelenz (DE); Robert Pinter, Lubeck (DE); Lena Gourmelon, Veldhoven (NL)

(72) Inventors: Guido Muesch, Erkelenz (DE); Robert Pinter, Lubeck (DE); Lena Gourmelon, Veldhoven (NL)

(73) Assignee: KONINKLIJKE PHILIPS ELECTRONICS N.V., EINDHOVEN (NL)

(21) Appl. No.: 13/876,251

(22) PCT Filed: Sep. 23, 2011
(86) PCT No.: PCT/IB11/54201

§ 371 Date: Mar. 27, 2013

(30) Foreign Application Priority Data
Oct. 1, 2010 (EP) 10185347.1

(51) Int. Cl. A61B 5/00; A61B 7/00; A61B 5/087

(52) U.S. Cl. CPC A61B 5/4818; A61B 5/087; A61B 7/003; A61B 5/7257
USPC 600/538; 600/529

(57) ABSTRACT

There is provided an apparatus for use in diagnosing the presence of obstructive sleep apnea in a patient, the apparatus comprising a processor configured to receive signals representing measurements of a patient's breathing obtained during a plurality o breathing cycles by the patient while the patient is awake, convert the signals into the frequency domain and to determine a value for at least one parameter based on an analysis of the frequency-domain converted signals in one or more frequency bands covering frequencies below 100 Hz.

### TECHNICAL FIELD OF THE INVENTION

The invention relates to an apparatus and method for collecting information, and in particular to an apparatus and method for collecting information from a patient that is awake and that can be used in diagnosing obstructive sleep apnea in the patient.

### BACKGROUND TO THE INVENTION

Obstructive sleep apnea (OSA) is a condition in which a subject experiences a decrease or complete stop in airflow while asleep, despite the subject continuing to try to breathe. These events occur when the muscles relax during sleep, causing soft tissue in the back of the throat to collapse and block the upper airway. This leads to partial reductions (known as hypopneas) and complete pauses (known as apneas) in breathing. An apnea event is defined as a cessation of airflow for at least 10 seconds during sleep. Hypopnea is defined as an abnormal respiratory event lasting at least 10 seconds with at least a 30 percent reduction in thoracoabdominal movement or airflow as compared to a baseline, with at least a 4 percent oxygen desaturation. Most apnea events last between 10 and 30 seconds, but some may persist for one minute or longer. This can lead to abrupt reductions in blood oxygen saturation, with oxygen levels falling as much as 40 percent or more in severe cases.

These apnea events cause the subject to wake briefly which restores normal breathing. As these apneas can occur tens or hundreds of times per night, the disruption caused results in the subject being excessively tired during the day.

A common measurement of sleep apnea is the apnea-hypopnea index (AHI). This is a number that represents the combined number of apneas and hypopneas that occur per hour of sleep. The following classification is frequently used:

AHI<5: No OSA/Healthy

5<AHI<15: Mild OSA

15<AHI<30: Moderate OSA

30<AHI Severe OSA

Generally, obstructive sleep apnea (OSA) is diagnosed in a sleep laboratory. However, most patients suffering from obstructive sleep apnea are never properly diagnosed since primary care physicians frequently deal with the symptoms of daytime fatigue and poor sleep by prescribing sleeping pills or similar medication. Physicians can be hesitant to send patients to a sleep laboratory immediately because of the high cost involved and the long waiting times. Usually patients are only sent when all other treatment attempts have failed and the patient keeps on complaining about bad sleep and daytime sleepiness. However, once a patient with suspected OSA is sent to a sleep laboratory, OSA is confirmed in around 85% of the cases.

OSA is diagnosed in a sleep laboratory with the help of “polysomnography” which is performed over one or more nights while the patient is asleep. Polysomnography can involve the use of an electroencephalogram (EEG), an electrocardiogram (ECG), an electroculogram (EOG), an electromyogram (EMG) and/or respiratory chest bands and the measurement of nasal airflow, blood oxygen levels and/or other physiological parameters. As a large number of sensors and devices are required for polysomnography, this procedure is not very comfortable or convenient for the patient.

Generally, the events of apnea and hypopnea in the polysomnography data are identified by a physician manually inspecting short intervals (roughly 30 seconds) of data, and individually rating the relevance of those intervals. Apnea events are characterized by the airflow through the patient's nasal passage stopping (or nearly stopping) while the thoracic and abdominal breathing movement continues. The number of identified events are counted and the average number of events per hour is used as an indicator of whether the patient has OSA and, if so, its severity.

However, a substantial amount of effort is required to scan the data covering a whole night in order to detect and count all apnea and hypopnea events, and to determine the AHI value for a patient.

Alternative techniques for diagnosing OSA have been proposed that involve the investigation of the snoring sounds of a patient. One such technique is described in “Investigation of Obstructive Sleep Apnea Using Nonlinear Mode Interactions in Nonstationary Snore Signals” by Ng et al., Annals of Biomedical Engineering, Vol. 37, No. 9, September 2009, pp. 1796-1806. However, this technique again requires the patient to attend a sleep laboratory and to be monitored while they are sleeping.

Therefore, there is a need for a more efficient method and apparatus for OSA screening, and that can be used while the patient is awake. Such a method and apparatus would allow more patients with suspected or possible OSA to be tested and would increase the number of patients with OSA that receive appropriate treatment for their condition.

One such method and apparatus that can be used while the patient is awake is disclosed in U.S. Pat. No. 6,942,626. However, applying this method to a larger data set could not provide reliable results.

In addition, the ‘snore analysis’ technique described above cannot be applied to data obtained from a patient that is awake since muscle tension persists in the upper airway of the awake patient, and this means that the signal processing techniques, which have been developed and optimized for signals obtained from sleeping patients, are not able to provide useful results.

### SUMMARY OF THE INVENTION

The invention provides a fast and comfortable OSA testing apparatus that is to be used while the patient is awake and a method of collecting information relating to obstructive sleep apnea while the patient is awake. The OSA test is targeted at detecting abnormalities of the upper airway in patients that have OSA and that influence the airflow during normal breathing while the person is awake.

Therefore, according to a first aspect of the invention, there is provided an apparatus for use in diagnosing obstructive sleep apnea in a patient, the apparatus comprising a processor configured to receive signals representing measurements of a patient's breathing obtained during a plurality of breathing cycles by the patient while the patient is awake, convert the signals into the frequency domain and determine a value for at least one parameter based on an analysis of the frequency-domain converted signals in one or more frequency bands covering frequencies below 100 Hz.

In one embodiment, the processor is configured to output the value for the at least one parameter to an operator of the apparatus.

In another embodiment, the processor is configured to determine whether the patient is likely to have obstructive sleep apnea based on the value of the at least one parameter and to output an indication of the likely presence or absence of obstructive sleep apnea in the patient to an operator of the apparatus.

Preferably, the processor is configured to determine whether the patient is likely to have obstructive sleep apnea based on a combination of values for a plurality of parameters.

The processor can be configured to determine a value for a first parameter by comparing the signals in a first frequency band covering frequencies below 100 Hz during exhalation to the signals in a second frequency band covering frequencies below 100 Hz during exhalation. Preferably, the first frequency band is 20-50 Hz, or, more preferably, 25-45 Hz, or, even more preferably, 30-40 Hz. Preferably, the second frequency band is 12-30 Hz, or, more preferably, 15-25 Hz, or, even more preferably, 18-22 Hz. Thus, in a preferred embodiment, the value for the first parameter is determined by comparing the signals during exhalation in the frequency band 30-40 Hz and the signals during exhalation in the frequency band 18-22 Hz.

In addition or alternatively to the first parameter described above, the processor can be configured to determine a value for a second parameter by comparing the signals in a third frequency band covering frequencies below 100 Hz during inhalation to the signals in the third frequency band during exhalation. Preferably the third frequency band is 0-20 Hz, or, more preferably, 0-15 Hz, or, even more preferably, 0-10 Hz.

In addition or alternatively to the first and second parameters described above, the processor can be configured to determine a value for a third parameter by comparing the signals in a fourth frequency band covering frequencies below 100 Hz during an inhalation or exhalation to a noise level above a threshold frequency during the inhalation or exhalation. Preferably, the fourth frequency band is 0-100 Hz and the threshold frequency for the noise level is 100 Hz or above, for example 200 Hz or 2000 Hz.

In a further embodiment, the processor is configured to determine a value for at least one further parameter based on a time domain analysis of the signals.

In yet another further embodiment, the processor is configured to determine a value for at least one further parameter, where the further parameter or parameters are selected from (i) the average length of a breathing cycle; and (ii) the ratio of the length of the inhalation to the length of the exhalation.

Preferably, the processor is configured to receive signals indicative of the rate of air flow during the plurality of breathing cycles by the patient while the patient is awake.

In one embodiment, the apparatus further comprises an air flow measuring device for measuring the flow rate of air over time during the plurality of breathing cycles by the patient while the patient is awake and for generating the signals indicative of the rate of air flow during the breathing cycles.

In an alternative embodiment, the processor is configured to receive signals indicative of the sound of the patient's breathing during the plurality of breathing cycles by the patient while the patient is awake. In a further embodiment, the apparatus further comprises a sound measuring device for measuring the sound of the air flow over time during the plurality of breathing cycles by the patient while the patient is awake and for generating the signals indicative of the sound of the patient's breathing.

In one embodiment, the processor is configured to convert the signals into the frequency domain by performing a respective Fast Fourier Transform, FFT, on the signals in each inhalation and exhalation part of the breathing cycle.

However, in a preferred embodiment, the processor is configured to convert the signals into the frequency domain by identifying the peak air flow during each inhalation and exhalation part of the breathing cycle and performing a Fast Fourier Transform, FFT, on the signals around the peak flows in each inhalation and exhalation part of the breathing cycle.

According to a second aspect of the invention, there is provided a method of determining or collecting information on a patient, the method comprising obtaining signals representing measurements of a patient's breathing during a plurality of breathing cycles by the patient while the patient is awake; converting the signals into the frequency domain; and determining a value for at least one parameter relevant to the diagnosis of obstructive sleep apnea based on an analysis of the frequency-domain converted signals in one or more frequency bands covering frequencies below 100 Hz.

In one embodiment, the method further comprises the step of outputting the value for the at least one parameter. In an alternative embodiment, the method further comprises the step of combining the values of a plurality of parameters determined in the step of determining and outputting the result of the combination.

In preferred embodiments, the at least one parameter comprises:

(i) a comparison of the signals in a first frequency band covering frequencies below 100 Hz during exhalation to the signals in a second frequency band covering frequencies below 100 Hz during exhalation;
(ii) a comparison of the signals in a third frequency band covering frequencies below 100 Hz during inhalation to the signals in the third frequency band during exhalation; and/or
(iii) a comparison of the signals in a fourth frequency band covering frequencies below 100 Hz during an inhalation or exhalation to a noise level above a frequency threshold during the inhalation or exhalation.

In these preferred embodiments, the first frequency band is preferably 20-50 Hz, or, more preferably, 25-45 Hz, or, even more preferably, 30-40 Hz, the second frequency band is preferably 12-30 Hz, or, more preferably, 15-25 Hz, or, even more preferably, 18-22 Hz, the third frequency band is preferably 0-20 Hz, or, more preferably, 0-15 Hz, or, even more preferably, 0-10 Hz, the fourth frequency band is preferably 0-100 Hz and the frequency threshold is 100 Hz or above, for example 200 Hz or 2000 Hz.

Further embodiments can comprise the step of determining a value for at least one further parameter based on a time domain analysis of the signals. In these further embodiments, the at least one further parameter can comprise (i) the average length of a breathing cycle; and (ii) the ratio of the length of the inhalation to the length of the exhalation.

In some embodiments, the step of converting comprises performing a respective Fast Fourier Transform, FFT, on the signals in each inhalation and exhalation part of the breathing cycle. However, in alternative embodiments, the step of converting comprises identifying the peak air flow during each inhalation and exhalation part of the breathing cycle and performing a Fast Fourier Transform, FFT, on the signals around the peak flows in each inhalation and exhalation part of the breathing cycle.

According to a third aspect of the invention, there is provided a method of diagnosing obstructive sleep apnea in a patient, the method comprising performing the steps in the method described above on a patient that is awake and determining whether the patient has obstructive sleep apnea based on the value of the at least one parameter.

Thus, the invention provides an apparatus that can perform a fast, cost-effective and comfortable test on a patient that is awake in order to diagnose OSA and a method that provides information to a physician that allows them to determine if the patient has OSA.

### BRIEF DESCRIPTION OF THE DRAWINGS

Exemplary embodiments of the invention will be described in detail below with reference to the following drawings, in which:

FIG. 1 is a block diagram of an apparatus according to the invention;

FIG. 2 is a flow chart showing the functional steps in a method according to an embodiment of the invention;

FIGS. 3(a) and (b) are graphs illustrating the filtering process performed in a preprocessing step;

FIG. 4 illustrates the application of a sliding window FFT to only a part of each inhalation or exhalation segment around the peak air flow; and

FIGS. 5(a) and (b) illustrate a typical frequency spectrum for a healthy patient and a patient with obstructive sleep apnea respectively.

### DETAILED DESCRIPTION OF THE PREFERRED EMBODIMENTS

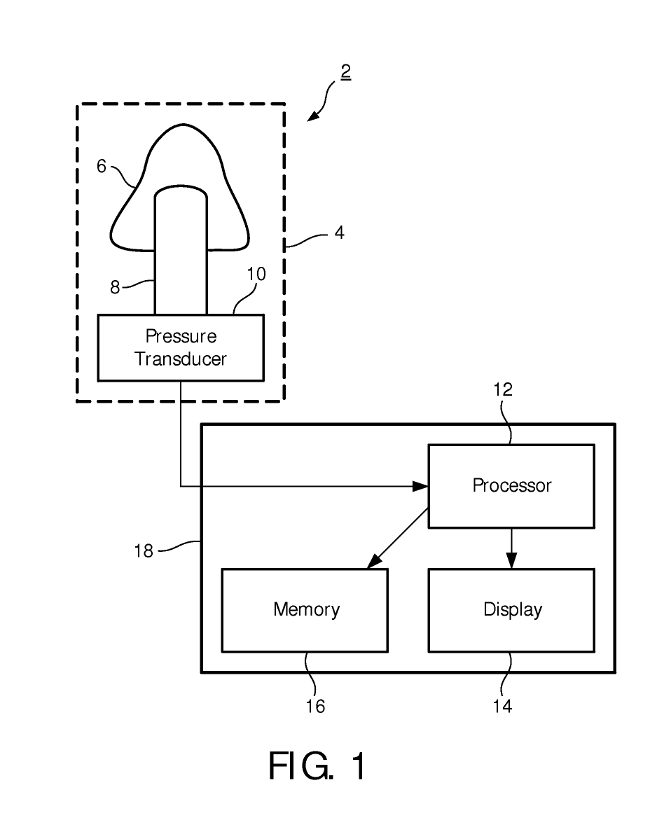

FIG. 1 shows an exemplary apparatus 2 according to the invention that can be used in the detection of obstructive sleep apnea in a patient based on data that is collected from the patient while they are awake.

In a preferred embodiment, the apparatus 2 comprises an air flow measuring device 4, such as a pneumotachograph, for providing measurements of the flow of air during inhalations and exhalations by a patient. As known, a pneumotachograph 4 comprises a nasal mask, facial mask or mouthpiece 6 that can be worn by the patient, a pneumotachometer 8 that is connected to the nasal mask, facial mask or mouthpiece 6, that measures the flow of air being inhaled and exhaled by the patient through the nasal mask, facial mask or mouthpiece 6 and provides an output in terms of a differential pressure, and a pressure transducer 10 that is connected to the pneumotachometer 8 and that converts the differential pressure output into an electrical signal, preferably digital samples.

The electrical signal is provided from the pressure transducer 10 in the pneumotachograph 4 to a processor 12 where it is processed to determine information that can be used by a physician to determine whether the patient has a sleep-related breathing disorder, such as obstructive sleep apnea (OSA). The processor 12 is connected to a display 14 that provides a visual indication of the result of the processing (such as the information to be used by the physician in diagnosing the patient, and/or, in some implementations of the invention, an indication of whether the patient has OSA or other breathing disorder). The processor 12 is also connected to a memory 16 that can store the electrical signals output from the pneumotachograph 4 prior to processing by the processor 12, as well as any result or results of the processing performed by the processor 12 on the electrical signals.

In this illustrated embodiment, the processor 12, display 14 and memory 16 are contained in a processing unit 18 that forms a separate unit to the pneumotachograph 4. In this case, the electrical signals from the pneumotachograph 4 can be provided to the processor 12 in the processing unit 18 via a connecting wire, wirelessly using WiFi, Bluetooth, etc., or by any other suitable means. However, in alternative implementations, the pneumotachograph 4 and processing unit 18 can be provided within a single housing. In either case, the apparatus 2 is preferably implemented as a lightweight device that can be easily held or worn by the patient during a testing procedure without causing the patient undue discomfort.

Although not shown in FIG. 1, it will be appreciated that the apparatus 2 (and in particular the processing unit 18) may include additional components, such as a user interface for allowing a user of the apparatus 2 to input commands and/or patient-specific data to the processor 12 and/or an internal power supply such as a battery if the apparatus 2 is to be operated independently of an external power supply.

In alternative embodiments, the pneumotachograph 4 can be replaced by an alternative means that can provide measurements of air flow, such as a nasal cannula.

FIG. 2 is a functional diagram illustrating the operations performed by or in the apparatus 2 according to the invention. In a first step 32, electrical signals representing the air flow to and from the patient's lungs during breathing while the patient is awake are acquired from the pneumotachograph 4. The electrical signals preferably comprise digital samples representing the magnitude (i.e. rate) of the air flow at respective sampling instants. As suggested above, the first step 32 is performed while the patient is awake.

The air flow rate samples are passed to the processor 12 where they are processed to provide information relating to the breathing condition of the patient. In some embodiments this information is presented to a physician to assist the physician in diagnosing obstructive sleep apnea. In other embodiments, the processor can further process the information to provide an indication of whether the patient has obstructive sleep apnea, which can be output by the apparatus 2 to an operator (such as a physician), for example using the display 14.

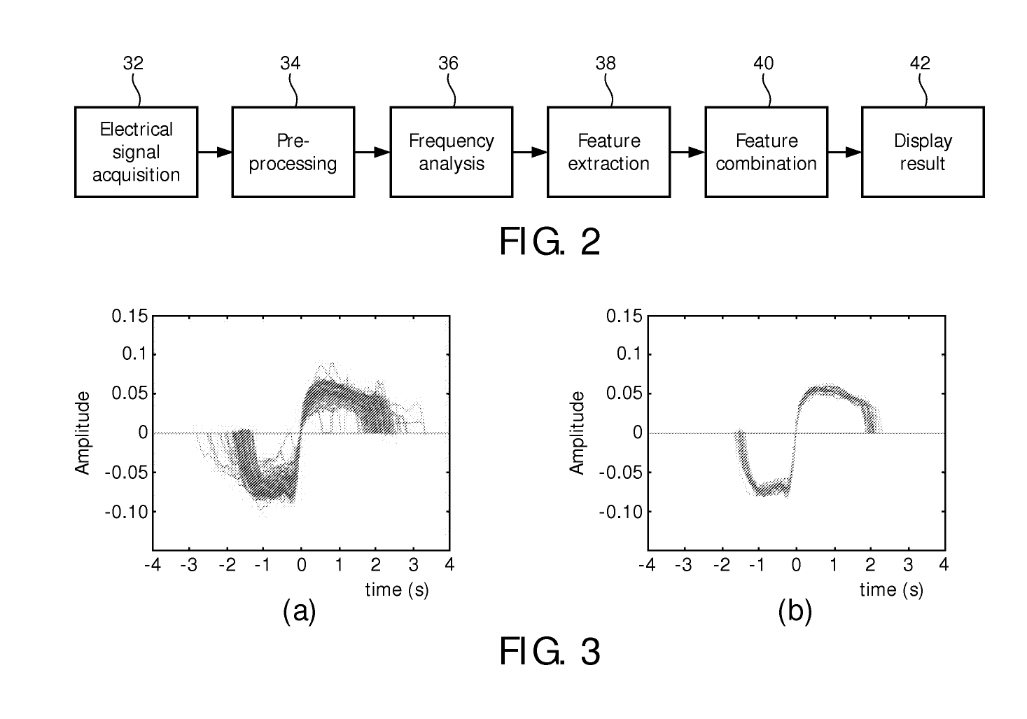

It has been found that the raw sample data can contain artifacts (for example see FIG. 3(a) discussed below), which can affect the quality of the analysis performed in subsequent processing steps. Therefore, it is desirable to provide a step that assesses the quality of the raw sample data and selects a subset of the data for one or more breathing cycles that are to be used in the subsequent processing steps. Thus, the first processing step performed by the processor 12 is a pre-processing step (step 34 in FIG. 2) in which the raw sample data is processed to identify N breathing cycles (with a single breathing cycle comprising a consecutive inhalation and exhalation) that are to be used in subsequent processing steps. Preferably, the N breathing cycles selected are those breathing cycles that best fit a mean breathing cycle for the patient. In preferred embodiments N is 12, although N can take any positive integer value.

The selection of the N breathing cycles is preferably performed as follows. Firstly, the raw sample data is separated into individual breathing cycles, and preferably individual inhalation and exhalation segments. The transition points between each inhalation and exhalation (i.e. where the patient starts to exhale after inhaling and exhaling after inhaling) can be easily identified from the zero-crossings in the sample data.

Next, the breathing cycles or individual inhalation and exhalation segments are filtered using one or more criteria, for example, a minimum length, the deviation from a mean length (in total and also separately for inhalation and exhalation segments) and deviation from a mean shape. The N cycles or segments best meeting the required criteria are then selected for further analysis by the processor 12.

FIGS. 3(a) and 3(b) illustrate the filtering process performed in the preprocessing step 34. FIG. 3(a) is a graph plotting the sample data obtained from a patient, with the data split into individual breathing cycles. The transition between inhalation and exhalation in each breathing cycle is plotted at the origin of the graph. The samples with a negative amplitude represent air flowing into the patient's lungs (i.e. during inhalation), and the samples with a positive amplitude represent air flow out of the patient's lungs (i.e. during exhalation). Thus, it can be seen that the air flow in many of the breathing cycles follows a generally regular pattern, but there are a number of breathing cycles in which the air flow varies considerably from the regular pattern (i.e. they contain artifacts). The filtering step described above results in the selection of N=12 breathing cycles fitting a mean breathing cycle for the patient, as shown in FIG. 3(b).

In one embodiment of the invention, in order to reduce the amount of time that a patient has to be attached to the testing apparatus 2, the processor 12 can perform the pre-processing step while the data is being collected, and can provide an indication to the patient or other user of the apparatus 2 that the test can be stopped once the data for N breathing cycles has been collected.

After the preprocessing step, the processor 12 performs a frequency analysis step 36 in which the sample data is converted into the frequency domain and a mean frequency spectrum is calculated. In particular, a sliding window Fast Fourier Transform (FFT) is applied to each individual breathing cycle to give a frequency spectrum.

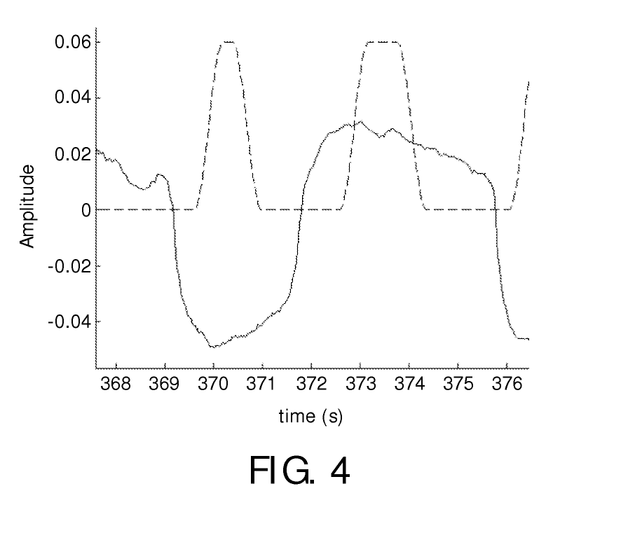

In some implementations, the sliding window FFT can be applied to each complete inhalation or exhalation segment. However, in preferred embodiments, the sliding window FFT is applied to only a part of each inhalation or exhalation segment around the peak air flow (i.e. where the air flow rate is at a local maximum). This preferred embodiment is illustrated in FIG. 4, in which the air flow samples are represented by the solid line (negative values again representing inhalation and positive values representing exhalation), and the dashed line indicates the samples to which the sliding window FFT is applied. Thus, it can be seen that the sliding window FFT is applied at and around the samples where the peak air flow occurs during each inhalation and exhalation. It has been found that this narrow sliding window approach provides a better data set for use in subsequent analysis by the processor 12.

In a particular embodiment, the width of the window is less than one second (so for example at a sample rate of 2600 Hz a FFT sliding window of width 2″=2048 is used, and for a sample rate of 26000 Hz a FFT sliding window of width 2″=16384 is used). The FFT window is then shifted by three-quarters of the FFT window length.

The N frequency-transformed breathing cycles are then averaged to provide separate mean frequency spectrums for inhalation and exhalation.

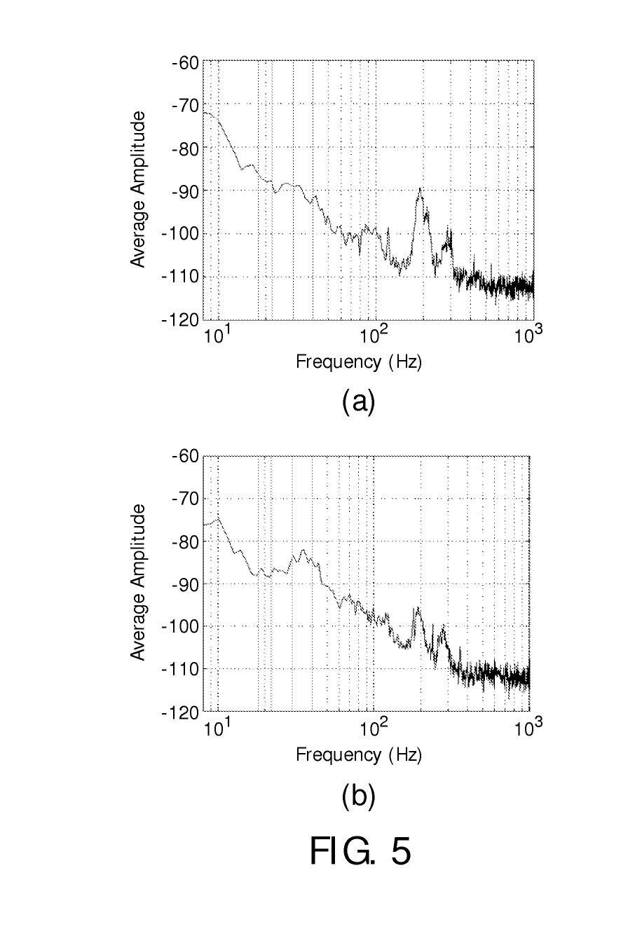

It has been found that the frequency spectrum obtained from air flow sample data for patients with a breathing disorder, such as obstructive sleep apnea, differs from the frequency spectrum obtained from healthy patients. For example, changes have been identified in certain frequency ranges or bands below 100 Hz, most notably the 18-22 Hz and 30-40 Hz frequency bands. These changes are illustrated in FIG. 5 which shows the mean exhalation frequency spectrum for a healthy patient (FIG. 5(a)) and a patient with obstructive sleep apnea (FIG. 5(b)). Thus, it can be seen, for example, that there is an elevation in the 30-40 Hz frequency band and a reduction in the 18-22 Hz frequency band for a patient with OSA compared to a healthy patient. Similar characteristics have been found in the mean inhalation frequency spectrum.

Thus, in accordance with the invention, the processor 12 extracts values for one or more parameters from the frequency spectrum or spectrums determined in the frequency analysis processing step 36. In particular, the value for at least one parameter is determined from the signals in one or more frequency bands covering frequencies that are below 100 Hz.

Various different parameters can be extracted in the feature extraction step 38 according to the invention.

One parameter that can be extracted is the difference between the mean exhalation frequency amplitude in a first frequency band, for example the range of 20-50 Hz, or, more preferably, 25-45 Hz, or, even more preferably, 30-40 Hz (denoted fₑₓ₃₀₋₄₀), and the mean exhalation frequency amplitude in a second frequency band, for example preferably the range of 12-30 Hz, or, more preferably, 15-25 Hz, or, even more preferably, 18-22 Hz (denoted fₑₓ₁₈₋₂₂). The parameter value can be given by fₑₓ₃₀₋₄₀-fₑₓ₁₈₋₂₂, and according to the observation described above, the value of the parameter for a healthy patient will generally be negative, whereas the value will generally be higher for a patient with OSA. Thus, the value of this parameter can be used by a physician or the apparatus 2 as an indicator as to whether the patient has OSA (perhaps by comparison to a threshold based on the parameter value(s) obtained for one or more healthy, non-OSA, patients). It will be appreciated by those skilled in the art that a value for a similar parameter can be obtained from the difference between the mean inhalation frequency amplitude in these or similar frequency ranges.

Furthermore, it will also be appreciated by those skilled in the art that the first and second frequency bands described above can be varied from the exemplary values given without substantially affecting the usefulness of the parameter in helping to diagnose OSA. For example, one or both of the most preferable frequency bands described above can be narrower (i.e. covering a smaller range of frequencies, for example 32-38 Hz and 19-21 Hz respectively), wider (i.e. covering a larger range of frequencies, for example 28-42 Hz and 17-23 Hz respectively) and/or shifted along the frequency spectrum (for example 28-38 Hz and 17-21 Hz respectively).

Another parameter that can be extracted is the difference between the mean exhalation frequency amplitude in a third frequency band, for example the range of 0-20 Hz, or, more preferably, 0-15 Hz, or, even more preferably, 0-10 Hz (denoted fₑₓ₀₋₁₀) and the mean inhalation frequency amplitude in the same or a similar frequency band, for example the range 0-20 Hz, or, more preferably, 0-15 Hz, or, even more preferably, 0-10 Hz (denoted fᵢₙ₀₋₁₀). The parameter value can be given by fₑₓ₀₋₁₀-fᵢₙ₀₋₁₀. The value of the parameter will be generally close to zero for a healthy patient, whereas the value will generally be higher for a patient with OSA. Thus, as with the first parameter above, the value of this parameter can be used by a physician or the apparatus 2 as an indicator as to whether the patient has OSA (perhaps by comparison to a threshold based on the parameter value(s) obtained for one or more healthy, non-OSA, patients).

As with the first parameter described above, it will be appreciated by those skilled in the art that the third frequency band described above can be varied from the exemplary value given without substantially affecting the usefulness of the parameter in helping to diagnose OSA. For example, the most preferable frequency band described above can be narrower (i.e. covering a smaller range of frequencies, for example 0-9 Hz), wider (i.e. covering a larger range of frequencies, for example 0-12 Hz) and/or shifted along the frequency spectrum (for example 2-12 Hz).

A further parameter that can be extracted is the difference between the mean frequency amplitude in the range 0-100 Hz for inhalation or exhalation (denoted fᵢₙ₀₋₁₀₀ or fₑₓ₀₋₁₀₀ as appropriate) and a ‘noise’ level at frequencies above 100 Hz. Again, these frequency bands can be varied from the exemplary value given without substantially affecting the usefulness of the parameter in helping to diagnose OSA (for example the threshold frequency for the noise level could be set higher than 100 Hz, for example 200 Hz or 2000 Hz).

Those skilled in the art will appreciate that the mean exhalation or inhalation frequency amplitude in a particular frequency band can be obtained from the output of the frequency analysis step 36 by averaging the amplitude of the frequency domain signal in the specified frequency band.

It will also be appreciated that the invention is not limited to the extraction of the specific parameters set out above, and that information useful for characterizing the breathing condition of a patient can be obtained from various other parameters that can be readily contemplated by those skilled in the art. In particular, and as discussed above, parameters can be extracted from frequency bands other than those specified above. Furthermore, it is not essential for the parameter or parameters to be based on the mean amplitude in a specified frequency band, since comparable results can be derived using other mathematical operations such as the area under the plot of the frequency spectrum in the frequency band or from the square of the amplitude.

One advantage of using the parameters described above (including the variations to the various frequency bands) is that, by comparing one part of the frequency spectrum for a patient to another part of the spectrum for the same patient, there is no need for the apparatus 2 to be calibrated for each new patient that is to be tested, which reduces the time required for the testing procedure.

In addition to extracting values for one or more parameters from the signals in the frequency domain, the processor 12 can extract values for other parameters from the time domain samples provided by the pneumotachograph 4 (whether the raw data or the data following the preprocessing step 34) during the feature extraction step 38.

For example, the processor 12 can extract time-domain features such as mean breathing cycle length and mean ratio between the length of the inhalation and length of the exhalation

Once the required parameter values have been extracted from the data, the processor 12 can either present the parameter values to a physician or other healthcare professional via the display 14 (or other visual output such as a printer-generated document) for use in assisting the physician to arrive at a diagnosis for the patient, or the processor 12 can perform a further processing step to combine the parameter values into a single useful score value.

In this feature combination step 40, the processor 12 can combine the extracted values of multiple parameters into a single score that can be used to assist in the diagnosis of a breathing disorder, as it has been found that a score based on the value of a number of the parameters described above is more useful in the reliable diagnosis of a breathing disorder than individual parameter values.

In one embodiment, the parameter values are combined linearly, for example:

Score, s=a+b·p₁+c·p₂+ . . . n·pₙ.

where p₁, p₂, . . . , pₙ denote the extracted values for respective parameters, and a, b, c, . . . , n are constant values, although other ways of combining the parameter values are within the scope of the invention, such as non-linear combinations or the use of decision trees.

In further embodiments, the score can also be based on other patient-related parameters, such as body-mass index (BMI), age, sex, Mallampati score, etc. which can be manually input to the apparatus 2 by the patient or operator.

In a particular embodiment, a score s_{OSA} useful in the assessment of a patient that might have OSA is given by

s_{OSA}=−3.21+0.13*p₁+0.13*p₂+0.14*p₃

where p₁ is the patient's BMI, p₂ is the difference between the mean amplitude between inhalation and exhalation in the frequency range 0-10 Hz, and p₃ is the difference in the mean amplitude in the frequency range 30-40 Hz and 18-22 Hz during exhalation. A positive value for s_{OSA} indicates that the patient is likely to have, or has OSA, whereas a negative value for s_{OSA} indicates that the patient is not likely to have OSA.

After the calculation of the score s, the result is displayed by the apparatus 2 (step 42 of the flow chart in FIG. 2). The score can be used by a physician or other healthcare professional to determine whether the patient has OSA or any other breathing disorder.

Alternatively, or in addition, the apparatus 2 can compare the determined score to one or more thresholds to determine an indication of whether the patient has a breathing disorder. In this case, the apparatus 2 can display the score and the indication (and optionally the parameter values used to calculate the score) to the operator of the apparatus 2.

As indicated above, if feature combination step 40 is omitted, the display step 42 can merely comprise displaying the value of the parameter or parameters determined in step 38. The parameter value or values can be noted by a physician or other healthcare professional and used to assist the physician in determining whether the patient has OSA. It will be appreciated that the physician or other healthcare professional can themselves derive a score as described above from the parameter value or values output by the apparatus 2, and optionally compare the score to one or more predetermined thresholds.

It will be appreciated by those skilled in the art that the signal processing method presented above is effectively the analysis of a signal resulting from the turbulence of the air being breathed in and out by the patient, which can be understood as a ‘sound’ overlying the air flow itself. Therefore, in alternative embodiments of the invention, it is possible for the signal data processed according to the invention to be obtained by a microphone or other sound sensor placed close to the patient (who is awake) while they breathe. These sound measurements can then be processed in a similar way to the air flow rate measurements described above, and the values for the appropriate parameters obtained.

There is therefore provided a method and apparatus for collecting information on a patient that can be used in diagnosing obstructive sleep apnea in the patient, where the information is collected while the patient is awake.

While the invention has been illustrated and described in detail in the drawings and foregoing description, such illustration and description are to be considered illustrative or exemplary and not restrictive; the invention is not limited to the disclosed embodiments.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. An apparatus (2) for determining one or more parameters for use in diagnosing the presence of obstructive sleep apnea in a patient while the patient is awake, the apparatus (2) comprising:
a processor (12) configured to receive signals representing measurements of a patient's breathing obtained during a plurality of breathing cycles by the patient, convert the signals into the frequency domain and to determine a value for at least one parameter based on an analysis of the frequency-domain converted signals in one or more frequency bands covering frequencies below 100 Hz,
wherein the processor (12) is configured to determine a value for a first parameter by comparing the signals in a first frequency band during exhalation to the signals in a second frequency band during exhalation, and/or determine a value for a second parameter by comparing the signals in a third frequency band during inhalation to the signals in the third frequency band during exhalation, and/or determine a value for a third parameter by comparing the signals in a fourth frequency band during an inhalation or exhalation to a noise level above a frequency threshold during the inhalation or exhalation.

2. An apparatus (2) as claimed in claim 1, wherein the processor (12) is configured to determine whether the patient is likely to have obstructive sleep apnea based on the value of the at least one parameter and to output an indication of the likely presence or absence of obstructive sleep apnea in the patient to an operator of the apparatus (2).

3. An apparatus (2) as claimed in claim 2, wherein the processor (12) is configured to determine whether the patient is likely to have obstructive sleep apnea based on a combination of values for a plurality of parameters.

4. (canceled)

5. An apparatus (2) as claimed in claim 1, wherein the processor (12) is configured to determine a value for the first parameter by comparing the signals during exhalation in the frequency band 30-40 Hz to the signals during exhalation in the frequency band 18-22 Hz.

6. (canceled)

7. An apparatus (2) as claimed in claim 1, wherein the processor (12) is configured to determine the value for the second parameter based on the signals in the frequency band 0-10 Hz.

8. An apparatus (2) as claimed in claim 1, wherein the processor (12) is configured to receive signals indicative of the rate of air flow during the plurality of breathing cycles by the patient.

9. An apparatus (2) as claimed in claim 1, further comprising:
an air flow measuring device (4) for measuring the flow rate of air over time during the plurality of breathing cycles by the patient while the patient is awake and for generating the signals indicative of the rate of air flow during the breathing cycles.

10. An apparatus (2) as claimed in claim 1, wherein the processor (12) is configured to receive signals indicative of the sound of the patient's breathing during the plurality of breathing cycles by the patient while the patient is awake.

11. An apparatus (2) as claimed in claim 1, wherein the processor (12) is configured to convert the signals into the frequency domain by identifying the peak air flow during each inhalation and exhalation part of the breathing cycle and performing a Fast Fourier Transform, FFT, on the signals around the peak flows in each inhalation and exhalation part of the breathing cycle.

12. A method for determining one or more parameters for use in diagnosing obstructive sleep apnea, comprising:
obtaining signals representing measurements of a patient's breathing during a plurality of breathing cycles by the patient while the patient is awake (32);
converting the signals into the frequency domain (36); and
determining a value for first parameter by comparing the signals in a first frequency band during exhalation to the signals in a second frequency band during exhalation; and/or
determining a value for a second parameter by comparing the signals in a third frequency band during inhalation to the signals in the third frequency band during exhalation; and/or
determining a value for a third parameter by comparing the signals in a fourth frequency band during an inhalation or exhalation to a noise level above a frequency threshold during the inhalation or exhalation;
wherein each of the first, second and third frequency bands covers frequencies below 100 Hz (38).

13. (canceled)

14. A method as claimed in claim 12, wherein the first frequency band is 30-40 Hz, the second frequency band is 18-22 Hz, the third frequency band is 0-10 Hz, the fourth frequency band is 0-100 Hz and the frequency threshold is 100 Hz or above.

15. A method of diagnosing obstructive sleep apnea in a patient, the method comprising:
performing the steps in the method according to claim 12 on a patient that is awake; and
determining whether the patient has obstructive sleep apnea based on the value of the at least one parameter.
